(19) United States
(12) Plant Patent Application Publication
(10) Pub. No.: US 2016/0249502 P1
(43) Pub. Date: Aug. 25, 2016

(54) Mango tree named 'R10/8'

(50) ;

(71) Applicant: Kenneth Rayner, Katherine (AU)

(72) Inventor: Kenneth Rayner, Katherine (AU)

(21) Appl. No.: 14/544,812

(22) Filed: Feb. 19, 2015

(51) Int. Cl. A01H 5/00

(52) U.S. Cl. PLT/159

(57) ABSTRACT

A new mango tree named ‘R10/8’, distinguished by its tendency to dwarfing and early maturing fruit.

### CROSS-REFERENCE TO RELATED APPLICATIONS

None

### GENUS AND SPECIES

Mangifera indica

### VARIETY DENOMINATION

‘R10/8’

### BACKGROUND AND SUMMARY OF THE VARIETY

The new mango tree ‘R10/8’ originated as a controlled cross of ‘Irwin’ (female parent, not patented) and an unknown male parent, likely ‘Kensington Pride’ (not patented). The initial cross was made at Katherine, Northern Territory, Australia, and seedlings resulting from the cross were planted in the same location. ‘R10/8’ was selected from among the seedlings for further observation due to its distinctive fruit and its tendency to dwarfing. In 2001, ‘R10/8’ was asexually propagated by grafting at Katherine, Northern Territory, Australia and was field planted the next year. ‘R10/8’ has since been observed to reproduce true to type over successive asexually propagated generations.

‘R10/8’ is distinguishable from its parents and other known varieties by the following characteristics:

‘R10/8’ is poly-embryonic, as compared to ‘Hayden’ (not patented) and ‘B74’ (“Calypso”, U.S. Plant Pat. No. 17,770) which are mono-embryonic.

‘R10/8’ is an early-maturing variety, as compared to ‘Kensington Pride’ which matures mid-season, and ‘Honey Gold’ (not patented) and ‘Parvin’ (not patented) which are late-maturing varieties.

### BRIEF DESCRIPTION OF THE PHOTOGRAPHS

FIG. 1 shows fruit of the ‘R10/8’ variety;

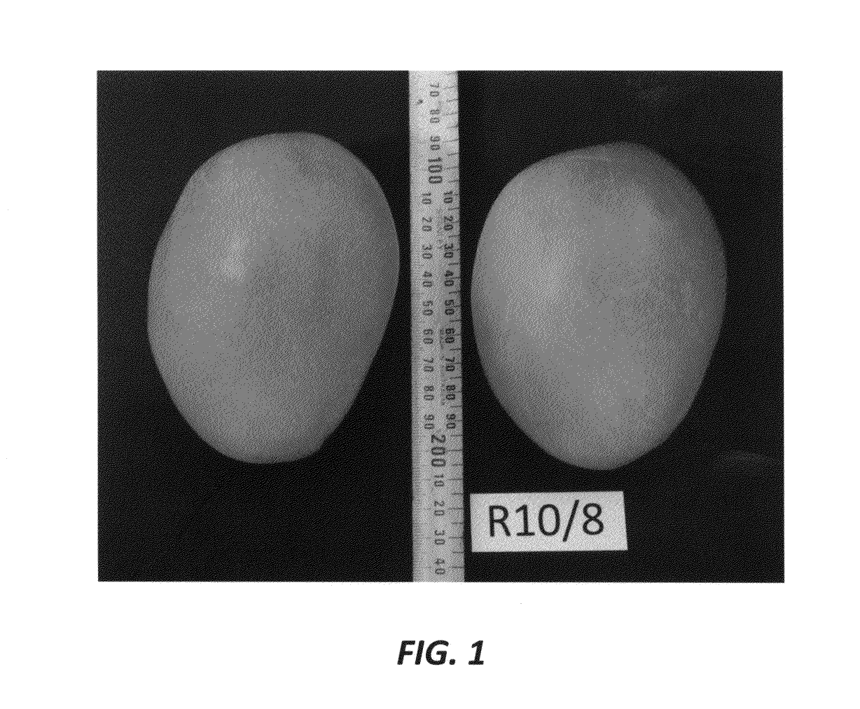

FIG. 2 shows fruit of the ‘R10/8’ variety; and,

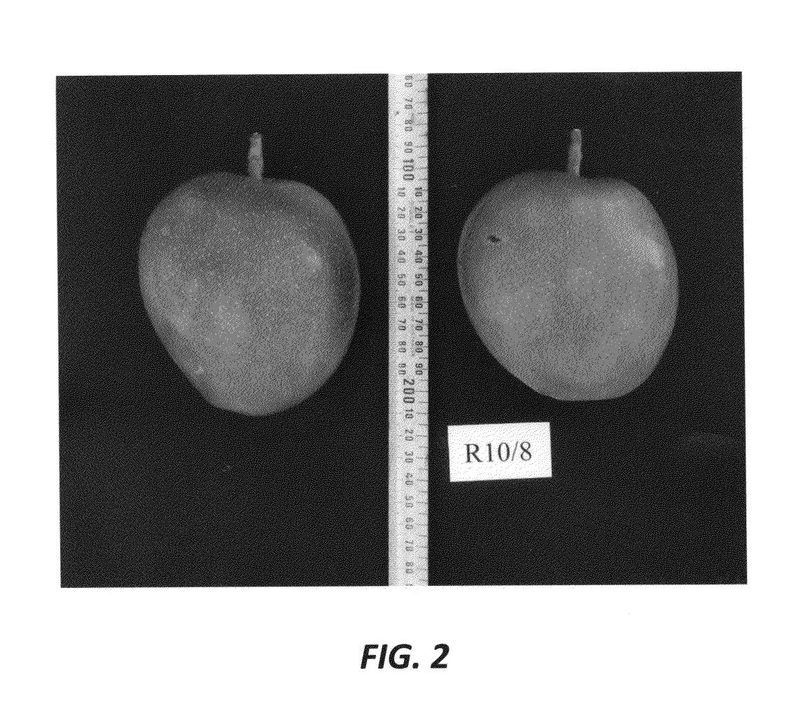

FIG. 3 shows a tree of the ‘R10/8’ variety.

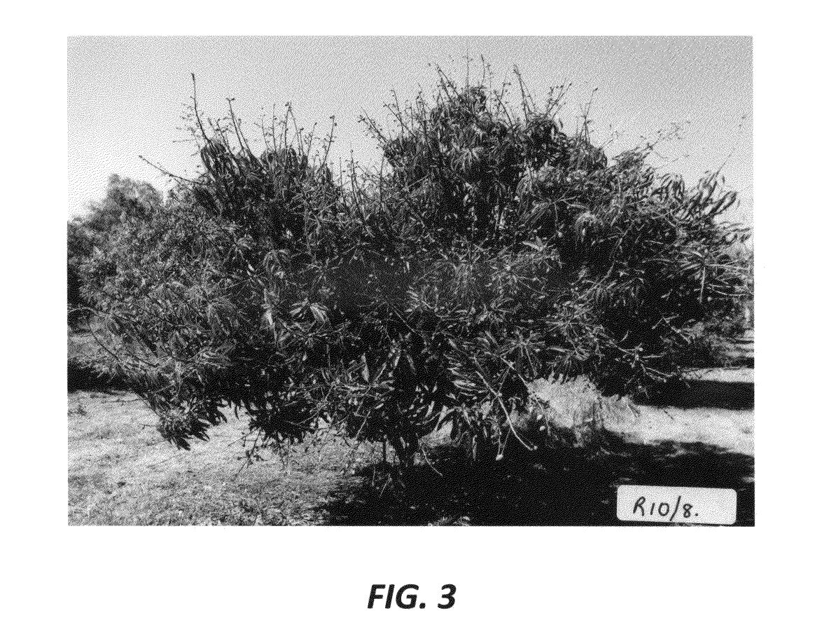

The colors of the claimed variety may vary with lighting conditions. Color characteristics of the variety should therefore be determined with reference to the observations described herein, rather than from these illustrations alone.

### DETAILED BOTANICAL DESCRIPTION

The following detailed botanical description is based on observations of trees planted in 2009 and grown on ‘RH34’ rootstock (not patented). Observations were recorded and photographs taken during the 2009 through 2012 growing seasons at Berry Springs, Northern Territory, Australia. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants of the new variety may vary from the stated average. Colors are described with reference to The Royal Horticultural Society Colour Chart (2005).
Tree:
   Vigor.—Low vigor.
   Height.—Small to medium, about 3.7 m.
   Spread.—about 4.3 m.
   Growth habit.—Spreading.
   Productivity.—Heavy, regular bearer.
   Trunk diameter.—20 cm at base.
   Bark texture.—Moderately rough.
   Bark color.—Brown.
Branches:
   Bark color.—Grey and green.
   Bark texture.—Smooth.
   Diameter.—13 cm to 16 cm (primary branches).
Inflorescence:
   Number of years to first flowering.—3 years.
   Regularity of flowering.—Every year.
   Form.—Panicle.
   Inflorescence position.—Apical.
   Axis growth habit.—Semi-erect.
   Shape.—Broadly pyramidal.
   Length.—40 cm.
   Width.—28 cm.
   Color.—Red.
   Peduncle length.—Short.
   Pubescence.—None.
   Presence of leafy bracts.—None.
   Density of flowers.—Very dense.
   Type of flower.—Pentamerous; male and hermaphrodite.
   Stamen.—Equal.
Leaves:
   Length.—22 cm.
   Width.—4.8 cm.
   Margin.—Entire, undulate.
   Pubescence.—None.
   Color.—young leaf Bronze to red.
   Color.—mature leaf Dark green.
   Leaf shape.—Lanceolate.
   Apex shape.—Acuminate.
   Base shape.—Cuneate.
   Petiole length.—4.8 cm.
   Petiole diameter.—3 mm.
   Petiole color.—Pale green.
   Leaf orientation.—Horizontal.
Fruit:
   Weight.—350 g to 430 g.
   Apex.—Rounded.
   Form of shoulder.—Rising, then rounded.
   Form of stalk cavity.—Shallow.
   Stalk attachment.—Vertical.
   Beak.—Present.
   Skin.—Tough, thin.
   Skin color of immature fruit.—Green; red blush when exposed to with direct sunlight.
   Skin color of mature fruit.—Bright yellow and orange with red blush.
   Flesh texture.—Very firm and juicy.
   Flesh color.—Yellow.
   Sweetness.—14 Brix and higher.
   Fiber.—Somewhat fibrous.
   Aroma.—Mild.
   Fruit per panicle.—1 to 3.
Stone:
   Length.—7.6 cm.
   Width.—4.4 cm.
   Depth.—1.6 cm.
   Venation.—Strong.
   Texture of stone fiber.—Soft.
   Adherence of fiber to stone.—Strong.
   Embryony.—Poly-embryonic.
   Storage.—Stores for about 12 days at ambient temperature.
   Susceptibility to bruising.—Slight.
   Susceptibility to wind.—None.
   Relative harvest maturity.—Early.

1. I claim a new and distinct variety of mango tree, substantially as illustrated and described herein.
